(19) United States
(12) Patent Application Publication
(10) Pub. No.: US 2011/0117142 A1
(43) Pub. Date: May 19, 2011

(54) METHOD FOR COATING TABLETS

(75) Inventors: Karl Kolter, Limburgerhof (DE); Nils Rottmann, Mannheim (DE); Maximilian Angel, Schifferstadt (DE)

(73) Assignee: BASF SE, Ludwigshafen (DE)

(21) Appl. No.: 13/002,179

(22) PCT Filed: Jul. 1, 2009
(86) PCT No.: PCT/EP2009/058278

§ 371 Date: Dec. 30, 2010

(30) Foreign Application Priority Data
Jul. 2, 2008 (EP) 08159504.3

(51) Int. Cl. A61K 9/00; A61K 47/32; A61K 8/02; C09D 139/06; A01C 1/06; B05D 3/02; A23P 1/08; A23P 1/00; A23L 1/00; A23L 1/29

(52) U.S. Cl. 424/400; 514/772.5; 524/548; 47/57.6; 427/385.5; 426/302; 426/305; 426/89

(57) ABSTRACT

The present invention relates to a polymeric composition for coating solid substrates (S) consisting of:
a) 0.5-90% by weight of polyethylene glycol with a weight-average molecular weight in the range from 1500 to 20 000 g/mol (component A),
b) 0.5-20% by weight of a water-soluble polyvinylpyrrolidone with a Fikentscher K value in the range from 12 to 90 (component B),
c) 0-95% by weight of a solvent (L), and
d) 0-70% by weight of one or more non-polymeric aids (component C).

The present invention relates to a process for coating substrates such as, for example, tablets, and to compositions for coating solid substrates such as tablets. The coating of solid dosage forms such as tablets with various film-forming polymers is a technique which has been widely used for years in modern drug formulation and tablet production. The use of so-called film coating has various objectives. Thus, for example, the color and appearance of the dosage form can be altered, a bitter taste can be masked, or the aim is to protect the active ingredient from light or improve the resistance to gastric juice.

Important requirements to be met by a film coating are that it is not tacky, adheres well to the substrate surface, is stable to mechanical stress and shows no fissuring on storage, and shows an excellent smoothness and gloss. In addition, it should be possible for the polymers of the film-coating solution to be prepared and dissolved in a solvent (such as water) quickly and easily. The film-coating solution should moreover have a low viscosity at a sufficiently high solids content in order to ensure good processing, e.g. in a spraying process.

Film-forming polymers have been employed for years for coating tablets, for example cellulose derivatives such as hydroxypropylcellulose, hydroxypropylmethylcellulose, methacrylic acid copolymers or else polyvinyl alcohol copolymers. The use of polymers such as polyethylene glycol and polyvinylpyrrolidone as film-forming components in coating compositions is also known.

Thus, GB 1 021 178 described in 1964 a protective tablet coating for wax-coated tablets composed of polyvinylpyrrolidone and polyethylene glycol. In the described process, these polymers are dissolved in an organic solvent (for example acetone, ethanol or trichloroethane) and sprayed onto the solid substrate. The solids content in these coatings is 3 to 18%, and the ratio of polyethylene glycol to polyvinylpyrrolidone in these mixtures is 1:1 to 9:1. The film coating described in GB 1 021 178 serves primarily to protect the underlying wax coating and is intended to increase the storage stability thereof. A gloss-increasing effect of the film coating is not described.

U.S. Pat. No. 4,060,598 (1975) describes a process for producing coated tablets in which an active ingredient-containing carrier is provided with a polymeric coating, where the coating is carried out by applying an aqueous composition with a water-insoluble and a water-soluble component. The resulting coating forms a matrix of water-insoluble porous resin and of a water-soluble polymeric material in the matrix pores. The coating serves to protect the active ingredient which is non-resistant to gastric juice, and may additionally take place in an aqueous coating process. Examples of the polymers in the water-soluble component mentioned in U.S. Pat. No. 4,060,598 are inter alia polyethylene glycol and polyvinylpyrrolidone. U.S. Pat. No. 4,060,598 also mentions preparations which comprise both polyethylene glycol and polyvinylpyrrolidone. However, these aqueous preparations also comprise in every case further polymeric components such as polyvinyl acetate and polyvinyl alcohol, plus filler and sugar.

WO 2006/102446 describes a multiply coated pharmaceutical preparation with modified dissolution behavior. In this case, an active ingredient, for example 5-aminosalicylic acid, in the core of the preparation is protected by a first coating from attack by gastric acid. The second outer coating is intended to protect the gastro-resistant first layer and may comprise various polymers, with polyvinylpyrrolidone and polyethylene glycol also being disclosed.

The composition of the invention is intended to make it possible to coat a substrate, preferably a solid carrier, preferably a pharmaceutical, cosmetic or agrochemical delivery form, seeds, dietary supplements and/or food products. The composition preferably consists of an aqueous solution of polyethylene glycol (component A) with a weight-average molecular weight M_{w} in the range from 1500 to 20 000 g/mol and a polyvinylpyrrolidone (component B) with a K value in the range from 12 to 90, and optionally further, non-polymeric aids (component C). The invention is further intended to provide an improved process for producing a coated, preferably solid substrate. If the substrate is a pharmaceutical delivery form, it may e.g. contain one or more drug compounds.

Known polymer compositions have the disadvantage when applied to substrates (such as tablets) of frequently producing a dull, unattractive and rough surface. In order to confer on such substrates a more attractive, glossy appearance and, for example, to make it easier for a patient to swallow the coated tablets, it is intended to coat substrates with an (optionally additional) smooth and glossy film. In order to achieve this aim, the intention is to provide, and apply to the substrate, a coating which produces or improves the gloss. It should be possible to carry out the process for applying the composition easily and preferably with an aqueous solution. The coating may also take place following a conventional film-coating process.

It has surprisingly been found that the coating, in particular of dull and rough substrates (such as film-coated tablets) with an aqueous solution of specific, water-soluble polyethylene glycols (PEG) and polyvinylpyrrolidones (PVP) has the effect of distinctly improving the gloss of the substrates (e.g. tablets).

The compositions of the invention can be prepared very easily and quickly by dissolving the components in a solvent (in particular water), have low viscosities and can be applied to solid substrates even in relatively high concentration of solids with a good application rate for example in conventional coating processes. The film coatings of the invention are distinguished not only by excellent gloss and smoothness but also by low tackiness and good storage stability.

The compositions of the invention have the effect of distinctly increasing the gloss by for example up to 285%. The combinations of polyvinylpyrrolidone and polyethylene glycol have the effect, as shown hereinafter, of improving the gloss distinctly more than solutions of the respective individual components.

The composition of the invention for coating solid substrates (S) comprises or consists preferably of:
a) 0.5-90% by weight of polyethylene glycol with a weight-average molecular weight in the range from 1500 to 20 000 g/mol (component A),
b) 0.5-20% by weight of a water-soluble polyvinylpyrrolidone with a Fikentscher K value in the range from 12 to 90 (component B),
c) 0-95% by weight of a solvent (L), and
d) 0-70% by weight of one or more non-polymeric aids (component C).

Preference is given to a composition for coating solid substrates (S),

such as pharmaceutical tablets containing, or preferably consisting of:
a) 5-20% by weight of polyethylene glycol with a weight-average molecular weight in the range from 1500 to 20 000 g/mol (component A),
b) 0.5-5% by weight of a water-soluble polyvinylpyrrolidone with a Fikentscher K value in the range from 12 to 90 (component B),
c) 65-95% by weight of a solvent (L), and
d) 0-10% by weight of one or more non-polymeric aids (component C).

A further embodiment relates to a composition for coating solid substrates containing, or preferably consisting of:
a) 5-20% by weight of polyethylene glycol with a weight-average molecular weight in the range from 4000 to 20 000 g/mol (component A),
b) 0.5-5% by weight of a water-soluble polyvinylpyrrolidone with a Fikentscher K value in the range from 12 to 30 (component B),
c) 75-95% by weight of a water as solvent (L).

Component B is preferably selected from one or more polyvinylpyrrolidones with a K value of from 12 to 90, preferably 12, 17, 30, 90, in particular 12, 17 or 30.

A further embodiment relates to a composition for coating solid substrates, where the solution has a polymer content in the range from 5 to 20, in particular 7 to 15, % by weight, and polyethylene glycol and polyvinylpyrrolidone are present in a ratio of from 15:1 to 7:1, but in particular about 9:1, by weight.

A composition which has proved particularly suitable for coating solid substrates containing or consisting of:
a) 8-10% by weight of polyethylene glycol with a weight-average molecular weight of 6000 g/mol (PEG 6000) (component A),
b) 0.5-2% by weight of a water-soluble polyvinylpyrrolidone with a Fikentscher K value of 17 (component B), and
c) 88-95% by weight of water.

The invention also relates to a process for producing coated solid substrates (such as tablets) in which at least one composition as described above is applied to a solid substrate (S).

This process can preferably include the following steps:
a) preparing a coating solution by mixing components A and B and the solvent L.
b) applying the coating solution by means of a suitable process to the substrate (S),
c) if appropriate drying the coated substrate by applying gas at a temperature in the range from 20° C. to 80° C., and
d) if appropriate polishing the coated and dried substrate.

The invention also relates to a process for producing coated solid substrates in which the substrate (S) is selected from: solid pharmaceutical, cosmetic and agrochemical delivery forms, seeds, dietary supplements and food products.

The invention also relates to a process for producing coated solid substrates in which the substrate (S) consists of a solid pharmaceutical dosage form (e.g. a tablet) which is already provided with at least one polymeric film coating.

The invention also relates to the use of a composition for the production of coated pharmaceutical, cosmetic or agrochemical delivery forms, of coated seeds, of coated dietary supplements or of coated food products.

A further aspect of the invention is the coated substrate which is coated with a composition as described above. This coated substrate may be based for example on a substrate (S) selected from: pharmaceutical, cosmetic and agrochemical delivery forms, seeds, dietary supplements and food products.

Water-soluble polyethylene glycols (PEG) with an average molecular weight M_{w} in the range from 1500 to 20 000, in particular from 1500 to 10 000, g/mol are employed in particular as component A. The following polyethylene glycols are preferably employed:
polyethylene glycol with an average molecular weight M_{w} of 1500 g/mol (PEG 1500)
polyethylene glycol with an average molecular weight M_{w} of 2000 g/mol (PEG 2000)
polyethylene glycol with an average molecular weight M_{w} of 4000 g/mol (PEG 4000)
polyethylene glycol with an average molecular weight M_{w} of 6000 g/mol (PEG 6000)
polyethylene glycol with an average molecular weight M_{w} of 20 000 g/mol (PEG 20 000)

Polyethylene glycol PEG 6000 is particularly preferably employed.

It is possible to employ as component B in particular polyvinylpyrrolidones (PVP) with a Fikentscher K value in the range from 12 to 90, preferably from 17 to 30, particularly preferably with K=17.

Polyvinylpyrrolidones (PVP) mean polymeric compounds which have a proportion of at least 50% polyvinylpyrrolidone monomer units and optionally further monomer units (such as, for example, vinyl acetate (VAc)). Polyvinylpyrrolidones virtually completely composed of vinylpyrrolidone units are preferred.

Preference is further given to compositions with polyvinylpyrrolidones (PVP) with a K value of 12, 17, 30, 90. The following polyvinylpyrrolidones are preferably employed:
Kollidon® 12 (BASF, Ludwigshafen DE) PVP with K=12
Kollidon® 17 PF (BASF, Ludwigshafen DE) PVP with K=17
Kollidon® 30 (BASF, Ludwigshafen DE) PVP with K=30
Kollidon® 90F (BASF, Ludwigshafen DE) PVP with K=90.

It is also possible to use copolymers of vinylpyrrolidone and vinyl acetate, e.g. the commercially available Kollidon® VA64 from BASF (PVP-vinyl acetate (VAc) copolymer).

The following polyvinylpyrrolidones are particularly preferably employed as component B in the compositions of the invention:
Kollidon® 17 PF (BASF, Ludwigshafen DE) PVP with K=17
Kollidon® 30 (BASF, Ludwigshafen DE) PVP with K=30.

Polyvinylpyrrolidones with K=17 are very particularly preferably employed.

The average molecular weight of the polyvinylpyrrolidone component B is preferably indicated in practice by the Fikentscher K value which can be determined relatively easily by measurements of the viscosity of the appropriate diluted polymer solutions. The K value is calculated from the relative viscosity ηᵣ by the Fikentscher equation:

$$\begin{matrix}K = 1000 \cdot k \\ = 1000 \cdot \frac{1.51 g {η}_{r} - 1 \pm \sqrt{1 + ( \frac{2}{c} + 2 + 1.51 g {η}_{r} ) \cdot 1.51 g {η}_{r}}}{150 + 300 \cdot c}\end{matrix}$$

with:
ηᵣ=dynamic viscosity of the solution/dynamic viscosity of the solvent
c=mass concentration of polymer in the solution in g/cm³

The Fikentscher K value thus represents a measure of the viscosity-average molecular weight.

The appropriate molecular weight ranges of the polyvinylpyrrolidones (PVP) employed, based on the weight-average molecular weight M_{w}, which can be ascertained for instance by light scattering measurements, are indicated below

PVP with a K value of 12: M_{w} 2000 to 3000 g/mol
PVP with a K value of 17: M_{w} 7000 to 11 000 g/mol
PVP with a K value of 30: M_{w} 44 000 to 54 000 g/mol
PVP with a K value of 90: M_{w} 1 000 000 to 1 500 000 g/mol.

The solvent (L) preferably used is water, in particular deionized water, but it is also possible to employ mixtures of water with other polar solvents such as mono- or polyhydric alcohols, alkyl halides, esters or ketones. Preferred polar organic solvents (L) are selected from the group: methanol, ethanol, n-propanol, n-butanol, isopropanol, chloroform or methylene chloride.

The compositions of the invention may optionally comprise as component C non-polymeric excipients as are usual as constituents of tablet coatings. It is possible to select as excipients in particular one or more of the following components:

### a) Coloring Components:

Colored pigments such as, for example, iron oxides and dyes in water-soluble or water-insoluble form, e.g. quinoline yellow lake, tartrazine lake, orange-yellow lake, FD&C yellow aluminum lake, cochineal red lake, erythrosine lake, azorubine lake, indigotine lake, erythrosine, brilliant black, patent blue, brilliant blue, cochineal red, orange-yellow color, amaranth, FD&C Blue No. 1, indigotine, beta-carotene, and pearlescent pigments

b) White pigments to increase the covering power of the coating, e.g. titanium dioxide, talc; mica; calcium carbonate
c) Non-stick agents, e.g. talc, magnesium stearate, glycerol monostearate;
d) Fillers such as, for example, calcium hydrogen phosphates;
e) Foam-inhibiting or destroying substances such as, for example, simethicone, octanol;
f) Surfactants to improve the wetting behavior and the spreading, e.g. sodium lauryl sulfate, sorbitan fatty acid esters or ethoxylated sorbitan fatty acid esters, ethoxylated esters of hydrogenated castor oil or ethoxylated fatty acid esters, sodium dioctylsulfosuccinate;
g) pH-regulating substances and buffers such as, for example, sodium citrate, citric acid, phosphate buffer, acetate buffer:

### h) Plasticizers;

i) Protective colloids;
j) Flavorings and/or odorants.

The compositions of the invention have the advantage that they usually do not require further aids such as plasticizers or surfactants which may cause negative side effects. In a preferred embodiment, no further excipients (component C) are present in the compositions.

The invention also relates to a dry mixture of components A and B and optionally C or granules of components A and B (and optionally C) which are prepared from this dry mixture and can then be used to prepare the glossing of film-coating solutions of the invention. The invention also relates to a kit consisting of the individual components of the composition which is then used by the user to prepare the coating composition.

The process for producing coated substrates may include for example the following specific steps:
a) preparing the coating solution by adding, with stirring, components A and B and optionally C to the solvent L,
or optionally mixing the solid components A and B and optionally C and subsequently dissolving the solid mixture in the solvent L,
or optionally dissolving or dispersing the components A, B and optionally C in parts of the solvent L and subsequently combining the solutions,
b) applying the coating solution by means of a suitable spraying process to the substrate
c) drying the coating, where the film coating is dried gradually by introducing air at a temperature in the range from 30° C. to 80° C.,
c) optionally carrying out a polishing step with the coated and dried substrates.

### Re Step a)

The compositions (film-coating solutions) are preferably prepared by slowly adding, with stirring, and completely dissolving components A and B and optionally C in the solvent L. It is not usually necessary to heat the solution during preparation.

In a further preferred embodiment, firstly components A and B and optionally C are mixed and possibly granulated. The composition is then prepared by adding the solid mixture with stirring to the solvent L.

### Re Step b)

It is possible in principle for all solid substrates to be coated with a polymer film by the coating process of the invention. Examples which can be coated are solid pharmaceutical, cosmetic and agrochemical delivery forms, seeds, dietary supplements and food products. The pharmaceutical dosage form to be coated can for example be in the form of a tablet, capsule, extrudate, pellet, granule, crystal or powder.

It is further possible to coat solid substrates with differently shaped surfaces, it being possible for the surface of the solid substrate to be for example curved, convex or concave. It is thus possible even with solid substrates with imprint to achieve a virtually complete covering of the imprint with a glossing coating by the composition of the invention. The solid substrates may have various shapes such as, for instance, circular, polygonal, oblong or football shape.

In a preferred embodiment, the substrate (S) to be coated is selected from solid pharmaceutical, cosmetic, agrochemical delivery forms, seeds, dietary supplements and food products.

The coating particularly preferably takes place on solid substrates which consist of a pharmaceutical dosage form such as a tablet which are already furnished with at least one polymeric coating (base coating).

Application of the process of the invention is in this case easy to carry out because the glossing film coating can be applied directly after the base coating or after the base coatings. No additional equipment is necessary, and the application time is short by comparison with the base coating process.

The base coatings in this case may serve for instance to protect the active ingredient, e.g. from water, oxygen, protons, chemical constituents of the coating, and from components of the stomach and bowel contents of for coloring the pharmaceutical dosage form.

Active ingredients of various areas of indication can be employed in the core of the pharmaceutical dosage form (or on the surface of the substrate), for example active pharmaceutical ingredients (human drugs and veterinary drugs), vitamins, carotenoids, nutraceuticals, dietary supplements, minerals and micronutrients. The active ingredients can be employed singly or in combination and have different pharmacological and physicochemical properties such as lipophilicity, solubility, particle size, particle structure and surface area.

The coating of the invention is preferably applied in a spray process which can be carried out in the coating devices suitable for this purpose. Examples mentioned are horizontal drum coaters, fluidized bed coaters, exchange value coaters and coating pans. The polymer solution is atomized by using for example a two-fluid nozzle.

It is also possible in this connection for the coating of the invention to be carried out immediately following a first coating of the substrate, for example of a pharmaceutical dosage form, and in the same coating equipment. It was possible to apply the solution of the invention to all tested colors and to all tested film-forming polymers.

It is possible to apply for example spray solutions with a solids content of up to 30% by weight. The optimal application rates are in the range from 0.2 to 2 mg/cm².

### Re Step c)

The drying and cooling time is preferably in the range from 2 to 20 minutes for a (product or core bed) temperature in the range from 20 to 50°, in particular 30 to 50° C. The spraying pressure is normally in the range from 1 to 3 bar.

### Re Step d)

The production of the coating may optionally be followed by a polishing step. The subsequent treatment (polishing) at low revolutions and low inlet air temperature has the effect with some formulations of further improving the gloss.

In a preferred embodiment of the invention, a solid pharmaceutical dosage form which is already coated with at least one base coating consisting of a polyethylene glycol/polyvinyl alcohol copolymer coated with a composition consisting of:
9% by weight of polyethylene glycol with a weight-average molecular weight of 6000 g/mol (PEG 6000) (component A),
1% by weight of the water-soluble polyvinylpyrrolidone with a Fikentscher K value of 17 (PVP-17) (component B), and
90% by weight of water (L)
   in a spraying process directly following a previous coating.

The following examples show the gloss-producing effect of various compositions of the invention on tablets which have already been film coated and have various shapes, colors and various base coatings.

### EXAMPLES

The components described in table 1 were employed:

TABLE 1
Starting materials

| Component | Designation | Description |
|---|---|---|
| Placebo cores | Placebo cores | 99.5% Ludipress ® LCE (BASF, |
| | | Ludwigshafen, DE); mixture of lactose |
| | | and Kollidon ® 30 (BASF, Ludwigshafen, |
| | | DE) as binder |
| | | 0.5% Mg stearate |
| Deionized water | Deionized water | Deionized water |
| A | PEG 1500 | polyethylene glycol with M_{w} = 1500 g/mol |
| A | PEG 2000 | polyethylene glycol with M_{w} = 2000 g/mol |
| A | PEG 4000 | polyethylene glycol with M_{w} = 4000 g/mol |
| A | PEG 6000 | polyethylene glycol with M_{w} = 6000 g/mol |
| A | PEG 20 000 | polyethylene glycol with M_{w} = 20 000 g/mol |
| B | PVP K 12 | polyvinylpyrrolidone with K = 12 |
| | | proprietary name Kollidon ® 12 (BASF, |
| | | Ludwigshafen DE) |
| B | PVP K 17 | polyvinylpyrrolidone with K = 17 |
| | | proprietary name Kollidon ® 17PF (BASF, |
| | | Ludwigshafen DE) |
| B | PVP K 30 | polyvinylpyrrolidone with K = 30 |
| | | proprietary name Kollidon ® 30 (BASF, |
| | | Ludwigshafen DE) |
| B | PVP K 90 | polyvinylpyrrolidone with K = 90 |
| | | proprietary name Kollidon ® 90F ® (BASF, |
| | | Ludwigshafen DE) |
| B | PVP-VAc copolymer | proprietary name Kollidon ® VA64 ® (BASF, |
| | | Ludwigshafen DE) |
| C | Hydroxypropylmethylcellulose | Pharmacoat ® 603 (Shin-etsu) |
| | (3 mPas) | |
| Base coating | PEG-PVA copolymer - white | polyethylene glycol-polyvinyl alcohol |
| type I | | copolymer granules with white pigment |
| | | (titanium dioxide, Kadin) |
| | | Kollicoat ® IR White |
| Base coating | PEG-PVA copolymer - yellow | polyethylene glycol-polyvinyl alcohol |
| type II | | copolymer granules with yellow dye |
| | | Kollicoat ® IR White + Sicovit ® Orange- |
| | | yellow (5%) |
| Base coating | PEG-PVA copolymer - red | polyethylene glycol-polyvinyl alcohol |
| type III | | copolymer granules with red colored |
| | | pigment |
| | | Kollicoat ® IR White + Sicovit ® Red (5%) |
| Base coating | PEG-PVA copolymer - carmine | polyethylene glycol-polyvinyl alcohol |
| type IV | | copolymer granules with carmine-colored |
| | | pigment |
| | | Kollicoat ® IR White + Carmine (5%) |
| Base coating V | PEG-PVA copolymer - brilliant | polyethylene glycol-polyvinyl alcohol |
| | blue | copolymer granules with brilliant blue- |
| | | colored pigment |
| | | Kollicoat ® IR White + Brilliant Blue (5%) |
| Base coating | PEG-PVA copolymer - black | polyethylene glycol-polyvinyl alcohol |
| type VI | | copolymer granules with black colored |
| | | pigment |
| | | Kollicoat ® IR + Sicovit ® Black (5%) |
| Base coating | PEG-PVA copolymer/PVA - | polyethylene glycol-polyvinyl alcohol |
| type VII | white | copolymer/polyvinyl alcohol with white |
| | | and red colored pigment |
| | | Kollicoat ® Protect + Kadin, titanium |
| | | dioxide and Sicovit ® Red |
| Base coating | Hydroxypropylmethylcellulose | Hydroxypropylmethylcellulose with white |
| type VIII | (HPMC), white | pigment (Kadin, titanium dioxide) |
| Red colored | Iron oxide | Iron oxide, proprietary name Sicovit ® Red |
| pigment | | (BASF, Ludwigshafen DE) |
| Yellow dye | Azo dye, C.I. Acid Yellow 3 | Proprietary name Sicovit ® Orange-yellow |
| | (EIIO) | (BASF, Ludwigshafen DE) |
| White pigment | Kadin | kadin |
| White pigment | Titanium dioxide | titanium dioxide |

Unless indicated otherwise, all percentage data relate to percent by weight, K refers to the Fikentscher K value, M_{w} refers to the weight-average molecular weight.

The following equipment was employed:
Automatic tablet-coating apparatus; horizontal drum coater of the Accela Cota 24″ type (from Manesty, Liverpool, GB)
High-shear mixer, Ultra Turrax (manufactured by IKA-Werke GmbH, Staufen DE)
Blade stirrer with drive
Magnetic stirrer
Glass equipment.

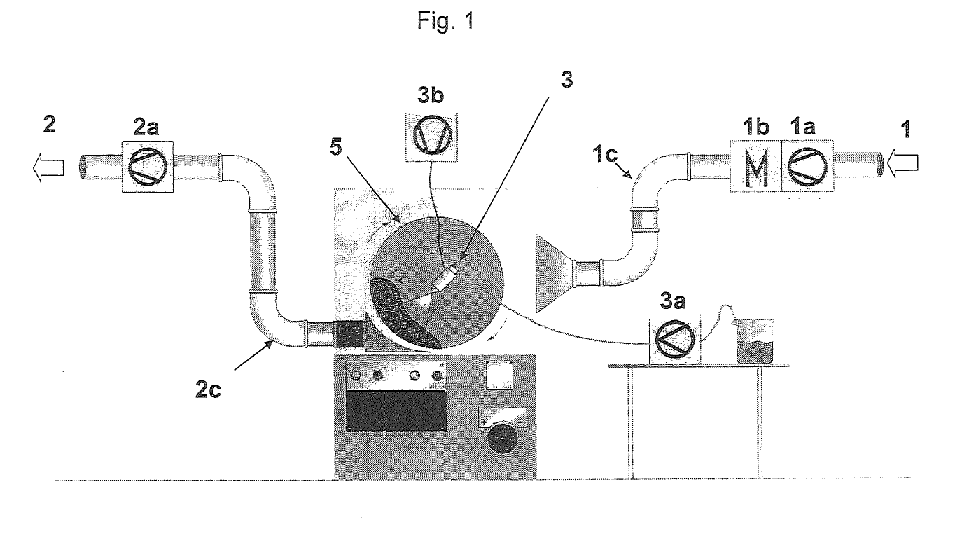

A sketch of the coating apparatus (Accela Cota 24″, Manesty) useful for the process is reproduced in FIG. 1. In the inflowing airstream (1), the inlet air rate (1a), the inlet air temperature (1b) and the inlet air moisture (1c) are determined. In the emerging air stream (2), the outlet air rate (2a) and the outlet air moisture (2c) is determined. The spray nozzle (3) is also depicted, it being possible to determine the spraying rate (3a) and spraying pressure (3b). The coating pan (4) rotates at the pan speed. A list of the parts and coating parameters depicted in FIG. 1 is reproduced below:
(1) Inlet air
(1a) Inlet air rate
(1b) Inlet air temperature
(1c) Inlet air moisture
(2) Outlet air
(2a) Outlet air rate
(2c) Outlet air moisture
(3) Spray nozzle
(3a) Spraying rate
(3b) Spraying pressure
(4) Coating pan.

The gloss was measured with a conventional gloss meter for curved surfaces, e.g. of the Novo-Curve™ 400 type (manufacturer: Elcometer Instruments, Manchester GB). The measurements were carried out at a measuring angle of 60° at room temperature (20° C.). The gloss is indicated dimensionlessly as gloss units [GU]. In each case, an average was formed from 10 measurements on 10 different tablets of the sample.

### Example 1

### Preparation of Coating Solutions (Film-Coating Solutions) for the Base Coating

a) Base Coating of Type II with Water-Soluble Dye

The mixtures used for the base coating were prepared by dissolving Kollicoat® IR White and the dye Sicovit® orange-yellow in deionized water. For this purpose, the components were dissolved or dispersed together in deionized water while stirring with a paddle stirrer. The solution was deaerated on a magnetic stirrer at low speed overnight. The polymer film consisted of 92.9% Kollicoat® IR White and 7.1% of the dye Sicovit® orange-yellow. The solution had a total solids content of 20%.

b) Base Coating of Type III-VI with Water-Insoluble Dye

Kollicoat® IR White was dispersed in a portion of the deionized water with stirring. Separately, the appropriate colored pigment was dispersed in another portion of the deionized water using a high-shear mixer (Ultra-Turrax). The two mixtures were combined with stirring and degassed by gentle stirring overnight.

c) Base Coating of Type VII and VII with Water-Insoluble Dye

The polymer solution used for the base coating was prepared by dissolving the polymer of type VII or type VIII and dispersing the colored pigments titanium dioxide, kadin, iron oxide (Sicovit® Red) in deionized water. For this purpose, the polymer was dissolved in a portion of the water, and the pigments were dispersed in another portion of the water using a high-shear mixer (Ultra Turrax). The two mixtures were combined with stirring by a paddle stirrer. The solution was deaerated on a magnetic stirrer at low speed overnight.

The polymer film of type VII consisted of 25% Kollicoat® Protect, 67% kadin, 2% titanium dioxide and 6% iron oxide (Sicovit® Red). The solution had a total solids content of 25%.

The polymer film of type VIII consisted of 75% hydroxypropylmethylcellulose, 10% kadin, 5% titanium dioxide and 10% polyethylene glycol 6000. The solution had a total solids content of 12%.

### Example 2

### Application of the Base Coating

The solutions or dispersions described in Examples 1a) to c) were applied separately to placebo cores composed of 99.5% Ludipress LCE® (BASF) and 0.5% magnesium stearate in a tablet-coating apparatus (24″ Accela Cota) under the application conditions detailed below. The spraying nozzle used was a conventional nozzle (model 930, manufacturer: Düsen-Schlick, Untersiemau DE) with a 1 mm bore. A sketch of the coating apparatus (Manesty 24″ Accela Cota) is reproduced in FIG. 1.

### Conditions of Application Process for the Base Coating:

| | | |
|---|---|---|
| spraying pressure | 2.0 | bar |
| pattern air pressure | 1.0 | bar |
| drum speed | 15 | rpm |
| inlet air rate | 60 | L/s |
| outlet air rate | 110 | L/s |
| inlet air temperature | 62° | C. |
| outlet air temperature | 45° | C. |
| product temperature | 37° | C. |
| spraying rate | 25 | g/min |
| drying/cooling time | 10 | min |
| application rate | 4.5 | mg/cm² |
| batch size | 5 | kg |

Following this process, a sample of at least 10 tablets was taken for gloss measurement. The gloss measurement on the film tablets was carried out with a conventional optical measuring apparatus (Novo-Curve™ type 400) at a measuring angle of 60° at room temperature. The gloss is indicated as usual dimensionlessly as gloss units [GU].

### Example 3

### Preparation of the Compositions for the Gloss Coating

The various compositions (film-coating solutions) were prepared by slowly adding and completely dissolving the polyethylene glycol and the polyvinylpyrrolidone (or the polyvinylpyrrolidone-polyvinyl acetate copolymer) in deionized water. The solutions were stirred with a magnetic stirrer during the preparation. Heating of the solution during the preparation was unnecessary. The polymer content of the compositions (film-coating solutions) was preferably 10%. Various polyethylene glycol and polyvinylpyrrolidone (or polyvinylpyrrolidone copolymer) were employed and tested in various ratios of amounts.

### Example 4

### Application of the Composition (of the Glossing Film Coating)

Directly after cooling of the base-coated film-coated tablets from example 2, the compositions prepared in example 3 (film-coating solutions) were applied in the suitable coating apparatus (24″ Accela Cota) under the application conditions detailed below. At least 10 tablets were drawn as samples directly after the application process and after the polishing process, and the gloss thereof was determined. Details of the polishing process are likewise mentioned below. The gloss measurement on the film-coated tablets was carried out with a conventional optical measuring apparatus (Novo-Curve™ type 400) at a measuring angle of 60° at room temperature. The gloss is indicated as usual dimensionlessly as gloss units [GU].

### Conditions of Coating Process:

| | | |
|---|---|---|
| spraying pressure | 2.0 | bar |
| pattern air pressure | 1.0 | bar |
| drum speed | 10 | rpm |
| inlet air rate | 60 | L/s |
| outlet air rate | 110 | L/s |
| inlet air temperature | 62° | C. |
| outlet air temperature | 45° | C. |
| product temperature | 37° | C. |
| spraying rate | 10 | g/min |
| drying/cooling time | 5 | min |
| application rate | 0.5 | mg/cm² |
| batch size | 5 | kg |

Conditions of Polishing Process:

| Drum speed | 2-3 | rpm |
|---|---|---|
| Inlet air rate | — | |
| Outlet air rate | — | |
| Product temperature | 37° C.-25° C. | |
| Process time | 30 | min. |

### Example 5

### Detailed Description of an Exemplary Process

One kilogram of a 20% strength coating solution of type III in deionized water was prepared for the base coating.

The degassed PEG-PVA copolymer (type III) solution was sprayed in a tablet-coating apparatus (horizontal drum coater of Accela Cota type 24″, Manesty) onto 5 kg of placebo cores (circular, curved, diameter 9 mm) of the following composition:

| | |
|---|---|
| Ludipress LCE | 99.5% by weight |
| Magnesium stearate | 0.5% by weight |

### Spraying Conditions:

| | | |
|---|---|---|
| spraying pressure | 2.0 | bar |
| pattern air pressure | 1.0 | bar |
| drum speed | 15 | rpm |
| inlet air rate | 60 | L/s |
| outlet air rate | 110 | L/s |
| inlet air temperature | 62° | C. |
| outlet air temperature | 45° | C. |
| product temperature | 37° | C. |
| spraying rate | 25 | g/min |
| drying/cooling time | 5 | min |
| application rate | 4.5 | mg/cm² |
| spraying time | 28 | min |

After the base coating, all the tablets were coated with a uniform, homogeneous, carmine-colored film. At least 10 tablets were taken as sample for the measurement of gloss.

The coating solution (glossing film-coating solution) consisted of nine parts of PEG 6000 and one part of Kollidon® 17, which were incorporated into 90 parts of water with stirring. The solid substances were slowly added and were completely dissolved after about ten minutes.

After the drying of the base-coated tablets in the coating apparatus (24″ Accela Cota) 170 g of the clear coating solution (glossing film-coating solution) were applied at a core bed temperature of about 36° C. under the conditions indicated below.

### Spraying Conditions:

| | | |
|---|---|---|
| spraying pressure | 2.0 | bar |
| pattern air pressure | 1.0 | bar |
| drum speed | 15 | rpm |
| inlet air rate | 60 | L/s |
| outlet air rate | 110 | L/s |
| inlet air temperature | 62° | C. |
| outlet air temperature | 45° | C. |
| product temperature | 36° | C. |
| spraying rate | 10 | g/min |
| drying/cooling time | 5 | min |
| application rate | 0.5 | mg/cm² |

A sample of at least 10 tablets of the dried, gloss-coated film-coated tablets is likewise taken for measurement of gloss. The remaining film-coated tablets are cooled in the drum at low speed under the conditions indicated below (polishing process).

### Polishing Conditions:

| Drum speed | 2-3 | rpm |
|---|---|---|
| Inlet air rate | — | |
| Outlet air rate | — | |
| Product temperature | 37° C.-25° C. | |
| Process timer | 30 | min. |

Following the polishing phase, the cooled film-coated tablets are moved from the drum. The gloss is determined for ten tablets.

The measurement of gloss was carried out as described in example 1.

### Example 6

### Comparison of Various Compositions for the Gloss Coating

For the base coating, solution of type II was prepared as described in example 1 and applied as described in example 2 to circular, curved placebo cores (99.5% Ludipress® LCE (BASF); 0.5% Mg stearate) with a diameter of 9 mm.

The compositions (film-coating solution) for the gloss coating were prepared as described in example 3, and the coating was carried out as described in example 4, employing in each case the PEG and PVP mentioned in tables 2 a) to g) below, in the stated ratio.

The measurement of gloss was carried out as described in example 1. The glosses [GU] of the corresponding film-coating solutions on circular, curved placebo cores with a diameter of 9 mm are compiled in tables 2 a)-g).

TABLE 2 a)
Pure PEG solution

| | Without polishing step | With polishing step |
|---|---|---|
| PEG 2000 | 5.9 | 6.8 |
| PEG 4000 | 6.2 | 8.4 |
| PEG 6000 | 8.0 | — |
| PEG 20 000 | 4.9 | 6.2 |

TABLE 2 b)
Pure Kollidon ® solutions

| | Without polishing step | With polishing step |
|---|---|---|
| Kollidon ® 17 | 3.1 | — |
| Kollidon ® 30 | 4.4 | 4.8 |
| Kollidon ® VA 64 | 3.5 | — |

TABLE 2 c
PEG solutions with Kollidon ® 12 (BASF)

| | Ratio (PEG/PVP) 18:1 | | Ratio (PEG/PVP) 9:1 | | Ratio (PEG/PVP) 5:1 | |
|---|---|---|---|---|---|---|
| | Without | With | Without | With | Without | With |
| | polishing | polishing | polishing | polishing | polishing | polishing |
| | step | step | step | step | step | step |
| PEG 1500 | 7.4 | 8.2 | 8.1 | — | 7.2 | — |
| PEG 2000 | 5.8 | 7.8 | 6.1 | 6.9 | 8.1 | 8.3 |
| PEG 4000 | 6.3 | 7.6 | 8.6 | 8.6 | 6.0 | — |
| PEG 6000 | 10.1 | — | 11.0 | 11.8 | 10.0 | 10.7 |
| PEG 20 000 | 4.9 | 5.8 | 3.9 | 5.1 | 6.9 | 7.9 |

TABLE 2 d
PEG solutions with Kollidon ® 17 (BASF)

| | Ratio (PEG/PVP) 18:1 | | Ratio (PEG/PVP) 9:1 | | Ratio (PEG/PVP) 5:1 | |
|---|---|---|---|---|---|---|
| | Without | With | Without | With | Without | With |
| | polishing | polishing | polishing | polishing | polishing | polishing |
| | step | step | step | step | step | step |
| PEG 1500 | 8.0 | 8.4 | 8.6 | 8.8 | 8.0 | 7.2 |
| PEG 2000 | 7.9 | 8.2 | 9.2 | 10.1 | 6.8 | 7.4 |
| PEG 4000 | 9.5 | — | 10.6 | — | 10.0 | 7.9 |
| PEG 6000 | 8.7 | 9.9 | 11.9 | 12.3 | 10.9 | 9.8 |
| PEG 20 000 | — | — | 6.8 | 8.9 | — | — |

TABLE 2 e
PEG solutions with Kollidon ® 30 (BASF)

| | Ratio (PEG/PVP) 18:1 | | Ratio (PEG/PVP) 9:1 | | Ratio (PEG/PVP) 5:1 | |
|---|---|---|---|---|---|---|
| | Without | With | Without | With | Without | With |
| | polishing | polishing | polishing | polishing | polishing | polishing |
| | step | step | step | step | step | step |
| PEG 1500 | 7.9 | — | 9.6 | 9.8 | 8.0 | — |
| PEG 2000 | — | — | 8.4 | — | — | — |
| PEG 4000 | 9.8 | 10.6 | 8.1 | 8.4 | 9.0 | — |
| PEG 6000 | 8.1 | 9.4 | 9.1 | 10.1 | 8.2 | 8.7 |
| PEG 20 000 | 6.2 | 7.3 | 5.9 | 6.8 | 7.4 | 8.2 |

TABLE 2 f
PEG solutions with Kollidon ® 90 (BASF)

| | Ratio (PEG/PVP) 18:1 | | Ratio (PEG/PVP) 9:1 | | Ratio (PEG/PVP) 5:1 | |
|---|---|---|---|---|---|---|
| | Without | With | Without | With | Without | With |
| | polishing | polishing | polishing | polishing | polishing | polishing |
| | step | step | step | step | step | step |
| PEG 1500 | — | — | 12.1 | — | — | — |
| PEG 2000 | — | — | 10.0 | — | — | — |
| PEG 4000 | 8.0 | 8.6 | 10.3 | 11.6 | 9.2 | — |
| PEG 6000 | 10.1 | — | 9.7 | — | 9.0 | — |
| PEG 20 000 | 4.9 | 5.4 | 8.0 | — | 5.9 | 8.0 |

TABLE 2 g
PEG solutions with Kollidon ® VA 64 (BASF)

| | Ratios (PEG/PVP) 18:1 | | Ratio (PEG/PVP) 9:1 | | Ratio (PEG/PVP) 5:1 | |
|---|---|---|---|---|---|---|
| | Without | With | Without | With | Without | With |
| | polishing | polishing | polishing | polishing | polishing | polishing |
| | step | step | step | step | step | step |
| PEG 1500 | — | — | 6.4 | — | — | — |
| PEG 2000 | — | — | 3.8 | 7.6 | — | — |
| PEG 4000 | 6.2 | 7.9 | 9.3 | — | 9.1 | 10.4 |
| PEG 6000 | 7.7 | 7.6 | 10.2 | 10.3 | 8.9 | 9.6 |
| PEG 20 000 | — | — | 6.7 | 8.1 | — | — |

Application of an aqueous solution of PEG and PVP has the effect in every case of distinctly improving the gloss. This applies to all the tested combinations. The combinations of PVP and PEG have the effect of improving the gloss distinctly more than solutions of the individual components. The subsequent treatment (polishing) at low revolutions and low inlet air temperature has the effect of improving the gloss with some compositions. The composition for the gloss coating can be applied directly after the base coating. No additional apparatus is necessary, and the application time is short compared with the base-coating process.

### Example 7

### Comparison of the Gloss Coating on Various Colors and Base Coatings

For the base coatings, coating solutions of type I-VIII were prepared as described in example 1. Coatings were carried out on round, curved placebo cores (99.5% Ludipress® LCE (BASF); 0.5% Mg stearate) with a diameter of 9 mm as described in example 2.

The compositions for the gloss coating were prepared as described in example 3 from 9 parts of PEG 6000, one part of PVP Kollidon® 17 (BASF) and 90 parts of deionized water. The coating solution was applied as described in example 4 to the base-coated tablets described above.

The measurement of gloss was carried out as described in example 1. The glosses of the coated tablets are indicated in table 3.

TABLE 3
Gloss coatings on base coatings of various colors and polymers

| | | Gloss | | Gloss | |
|---|---|---|---|---|---|
| | | without | Increase | with | Increase |
| | Gloss of the | polishing | in gloss | polishing | in gloss |
| Base coating | base coating | step | [%] | step | %] |
| Type I | 4.4 | 9.8 | 124.3 | 11.1 | 153.1 |
| Type II | 2.8 | 10.8 | 285.7 | 13.2 | 371.4 |
| Type III | 4.0 | 10.2 | 157.1 | — | — |
| Type IV | 2.5 | 8.2 | 230.6 | — | — |
| Type V | 3.0 | 9.3 | 206.3 | — | — |
| Type VI | 2.9 | 9.1 | 218.1 | — | — |
| Type VII | 4.0 | 11.3 | 182.5 | — | — |
| Type VIII | 4.2 | 10.1 | 140.5 | 10.3 | 145.2 |

Application of an aqueous composition (film-coating solution) composed of PEG and PVP has the effect in every case of distinctly improving the gloss. This applies to all tested combinations applicably of the substances used for the preparation. The solution is applicable to all tested colors and to all tested film-forming polymers and has the effect of increasing the gloss very greatly by up to 371.4%. The subsequent treatment (polishing) at low revolutions and low inlet air temperature has the effect of additionally improving the gloss for some compositions.

### Example 8

### Comparison of the Gloss Coating on Various Tablet Shapes

A coating solution of type II was prepared as described in example 1 for the base coating and was applied to placebo cores composed of 99.5% by weight Ludipress® LCE (BASF) and 0.5% by weight Mg stearate as described in example 2 to the following tablet shapes:
circular, curved, 6 mm,
circular, curved, 9 mm
oblong 8.5 mm

The compositions for the gloss coating were prepared as described in example 3 from 9 parts of PEG 6000, one part of PVP Kollidon® 17 (BASF) and 90 parts of water. The coating solution was applied as described in example 4 to the base-coated tablets described above.

The measurement of gloss was carried out as described in example 1. The glosses of the coated tablets are indicated in table 5.

TABLE 5
Gloss coatings on various tablet shapes

| | circular, curved, 6 mm | | circular, curved, 9 mm | | oblong 8.5 mm | |
|---|---|---|---|---|---|---|
| | Without | With | Without | With | Without | With |
| | polishing | polishing | polishing | polishing | polishing | polishing |
| | step | step | step | step | step | step |
| Without | Visually great | — | 2.8 | — | 3.2 | — |
| gloss coating | improvement | | | | | |
| | in gloss | | | | | |
| PEG 6000/ | Visually great | Visually great | 10.8 | 13.2 | 10.3 | 11.8 |
| Kollidon ® | improvement | improvement | | | | |
| 17 (9:1) | in gloss | in gloss | | | | |

Application of a glossing film-coating solution composed of PEG and PVP has the effect in every case of distinctly improving the gloss. The solution can be applied to all tested tablet shapes and has the effect of greatly increasing the gloss. The subsequent treatment (polishing) at low revolutions and low inlet air temperature has the effect of additionally improving the gloss.

1.-12. (canceled)

13. A composition for coating solid substrates (S) containing:
a) 5-20% by weight of polyethylene glycol with a weight-average molecular weight in the range from 1500 to 20 000 g/mol (component A),
b) 0.5-5% by weight of a water-soluble polyvinylpyrrolidone with a Fikentscher K value in the range from 12 to 90 (component B),
c) 65-95% by weight of water as solvent (L), and
d) 0-10% by weight of one or more non-polymeric aids (component C).

14. The composition for coating solid substrates according to claim 13 containing:
a) 5-20% by weight of polyethylene glycol with a weight-average molecular weight in the range from 4000 to 20 000 g/mol (component A),
b) 0.5-5% by weight of a water-soluble polyvinylpyrrolidone with a Fikentscher K value in the range from 12 to 30 (component B), and
c) 75-95% by weight of water as solvent (L).

15. The composition for coating solid substrates according to claim 13, wherein component B is selected from one or more polyvinylpyrrolidones with a K value of 12, 17, 30 or 90.

16. The composition for coating solid substrates according to claim 13, wherein the solution has a polymer content in the range from 5 to 20% by weight, and polyethylene glycol and polyvinylpyrrolidone are present in a ratio of about 9:1 by weight.

17. The composition for coating solid substrates according to claim 13, which consists of:
a) 5-10% by weight of polyethylene glycol with a weight-average molecular weight of 6000 g/mol (PEG 6000) (component A),
b) 0.5-2% by weight of a water-soluble polyvinylpyrrolidone with a Fikentscher K value of 17 (component B), and
c) 88-95% by weight of water.

18. The composition for coating solid substrates according to claim 17, wherein component A is in an amount from 8 to 10% by weight.

19. A process for producing coated solid substrates which comprises applying at least one composition containing:
a) 5-20% by weight of polyethylene glycol with a weight-average molecular weight in the range from 1500 to 20 000 g/mol (component A),
b) 0.5-5% by weight of a water-soluble polyvinylpyrrolidone with a Fikentscher K value in the range from 12 to 90 (component B),
c) 65-95% by weight of water as solvent (L), and
d) 0-10% by weight of one or more non-polymeric aids (component C) to a solid substrate (S).

20. The process for producing coated solid substrates according to claim 18, comprising the following steps:
a) preparing a coating solution by mixing components A and B and the solvent L.
b) applying the coating solution by means of a suitable process to the substrate (S),
c) optionally drying the coated substrate by applying gas at a temperature in the range from 20° C. to 80° C., and
d) optionally polishing the coated and dried substrate.

21. The process for producing coated solid substrates according to claim 18, wherein the substrate (S) is selected from: solid pharmaceutical, cosmetic and agrochemical delivery forms, seeds, dietary supplements and food products.

22. The process for producing coated solid substrates according to claim 18, wherein the substrate (S) consists of a solid pharmaceutical dosage form which is already provided with at least one polymeric film coating.

23. A coated substrate which is coated with a composition containing:
a) 5-20% by weight of polyethylene glycol with a weight-average molecular weight in the range from 1500 to 20 000 g/mol (component A),
b) 0.5-5% by weight of a water-soluble polyvinylpyrrolidone with a Fikentscher K value in the range from 12 to 90 (component B),
c) 65-95% by weight of water as solvent (L), and
d) 0-10% by weight of one or more non-polymeric aids (component C).

24. The coated substrate according to claim 22, wherein the substrate (S) is selected from pharmaceutical, cosmetic and agrochemical delivery forms, seeds, dietary supplements and food products.
